(19) United States
(12) Patent Application Publication
(10) Pub. No.: US 2021/0008156 A1
(43) Pub. Date: Jan. 14, 2021

(54) NOVEL RECOMBINANT BOTULINUM NEUROTOXINS WITH INCREASED DURATION OF EFFECT

(71) Applicant: MERZ PHARMA GMBH & CO. KGAA, Frankfurt am Main (DE)

(72) Inventors: Jürgen FREVERT, Berlin (DE); Fred HOFMANN, Potsdam (DE); Marcel JURK, Berlin (DE); Manuela LÓPEZ DE LA PAZ, Liederbach am Taunus (DE); Daniel SCHEPS, Potsdam (DE)

(21) Appl. No.: 16/755,848

(22) PCT Filed: Oct. 26, 2017
(86) PCT No.: PCT/EP2017/077427

§ 371 Date: Apr. 13, 2020

(51) Int. Cl. A61K 38/16; C12N 15/10; C12N 15/70

(52) U.S. Cl. CPC A61K 38/164; C12N 15/70; C12N 15/102

(57) ABSTRACT

The invention relates to novel recombinant single-chain precursor botulinum neurotoxins serotype A comprising at least one additional domain and least one amino acid modification of the heavy chain of the neurotoxin. The novel recombinant single-chain precursor botulinum neurotoxins further comprises at least one cleavage site for a protease selected from the group consisting of thrombin, HRV3C, Tobacco Etch Vims protease, enterokinase and factor Xa. The invention further relates to novel recombinant botulinum neurotoxins serotype A exhibiting an increased duration of effect.

### FIELD OF THE INVENTION

The invention relates to novel recombinant single-chain precursor botulinum neurotoxins serotype A comprising at least one additional domain and least one amino acid modification of the heavy chain of the neurotoxin. The novel recombinant single-chain precursor botulinum neurotoxins further comprises at least one cleavage site for a protease such as thrombin, HRV3C, Tobacco Etch Virus protease, enterokinase or factor Xa. The invention further relates to novel recombinant botulinum neurotoxins serotype A exhibiting an increased duration of effect. The invention also relates to methods for the manufacture of such recombinant botulinum neurotoxins. The invention further relates to pharmaceutical compositions comprising said recombinant neurotoxins.

### BACKGROUND OF THE INVENTION

Clostridium is a genus of anaerobe gram-positive bacteria, belonging to the Firmicutes. Clostridium consists of around 100 species that include common free-living bacteria as well as important pathogens, such as Clostridium botulinum and Clostridium tetani. Both species produce neurotoxins, botulinum toxin and tetanus toxin, respectively. These neurotoxins are potent inhibitors of calcium-dependent neurotransmitter secretion of neuronal cells and are among the strongest toxins known to man. The lethal dose in humans lies between 0.1 ng and 1 ng per kilogram of body weight.

Oral ingestion of botulinum toxin via contaminated food or generation of botulinum toxin in wounds can cause botulism, which is characterised by paralysis of various muscles. Paralysis of the breathing muscles can cause death of the affected individual.

Although both botulinum neurotoxin (BoNT) and tetanus neurotoxin (TxNT) function via a similar initial physiological mechanism of action, inhibiting neurotransmitter release from the axon of the affected neuron into the synapse, they differ in their clinical response. While the botulinum toxin acts at the neuromuscular junction and other cholinergic synapses in the peripheral nervous system, inhibiting the release of the neurotransmitter acetylcholine and thereby causing flaccid paralysis, the tetanus toxin acts mainly in the central nervous system, preventing the release of the inhibitory neurotransmitters GABA (gamma-aminobutyric acid) and glycine by degrading the protein synaptobrevin. The consequent overactivity in the muscles results in generalized contractions of the agonist and antagonist musculature, termed a tetanic spasm (rigid paralysis).

While the tetanus neurotoxin exists in one immunologically distinct type, the botulinum neurotoxins are known to occur in seven different immunogenic types, termed BoNT/A through BoNT/G. Most Clostridium botulinum strains produce one type of neurotoxin, but strains producing multiple toxins have also been described.

Botulinum and tetanus neurotoxins have highly homologous amino acid sequences and show a similar domain structure. Their biologically active form comprises two peptide chains, a light chain of about 50 kDa and a heavy chain of about 100 kDa, linked by a disulfide bond. A linker or loop region, whose length varies among different clostridial toxins, is located between the two cysteine residues forming the disulfide bond. This loop region is proteolytically cleaved by an unknown clostridial endoprotease to obtain the biologically active toxin.

The molecular mechanism of intoxication by TeNT and BoNT appears to be similar as well: entry into the target neuron is mediated by binding of the C-terminal part of the heavy chain to a specific cell surface receptor; the toxin is then taken up by receptor-mediated endocytosis. The low pH in the so formed endosome then triggers a conformational change in the clostridial toxin which allows it to embed itself in the endosomal membrane and to translocate through the endosomal membrane into the cytoplasm, where the disulfide bond joining the heavy and the light chain is reduced. The light chain can then selectively cleave so called SNARE-proteins, which are essential for different steps of neurotransmitter release into the synaptic cleft, e.g. recognition, docking and fusion of neurotransmitter-containing vesicles with the plasma membrane. TeNT, BoNT/B, BoNT/D, BoNT/F, and BoNT/G cause proteolytic cleavage of synaptobrevin or VAMP (vesicle-associated membrane protein), BoNT/A and BoNT/E cleave the plasma membrane-associated protein SNAP-25, and BoNT/C cleaves the integral plasma membrane protein syntaxin and SNAP-25.

Clostridial neurotoxins display variable durations of action that are serotype specific. The clinical therapeutic effect of BoNT/A lasts approximately 3 months for neuromuscular disorders and 6 to 12 months for hyperhidrosis. The effect of BoNT/E, on the other hand, lasts less than 4 weeks. The longer lasting therapeutic effect of BoNT/A makes it preferable for certain clinical use compared to the other serotypes, for example serotypes B₁, C₁, D, E, F, G. One possible explanation for the divergent durations of action might be the distinct subcellular localizations of BoNT serotypes. The protease domain of BoNT/A light chain localizes in a punctate manner to the plasma membrane of neuronal cells, co-localizing with its substrate SNAP-25. In contrast, the short-duration BoNT/E serotype is cytoplasmic. Membrane association might protect BoNT/A from cytosolic degradation mechanisms allowing for prolonged persistence of BoNT/A in the neuronal cell.

In Clostridium botulinum, the botulinum toxin is formed as a protein complex comprising the neurotoxic component and non-toxic proteins. The accessory proteins embed the neurotoxic component thereby protecting it from degradation by digestive enzymes in the gastrointestinal tract. Thus, botulinum neurotoxins of most serotypes are orally toxic. Complexes with, for example, 450 kDa or with 900 kDa are obtainable from cultures of Clostridium botulinum.

In recent years, botulinum neurotoxins have been used as therapeutic agents in the treatment of dystonias and spasms. Preparations comprising botulinum toxin complexes are commercially available, e.g. from Ipsen Ltd (Dysport®) or Allergan Inc. (Botox®). A high purity neurotoxic component, free of any complexing proteins, is for example available from Merz Pharmaceuticals GmbH, Frankfurt (Xeomin®).

Clostridial neurotoxins are usually injected into the affected muscle tissue, bringing the agent close to the neuromuscular end plate, i.e. close to the cellular receptor mediating its uptake into the nerve cell controlling said affected muscle. Various degrees of neurotoxin spread have been observed. The neurotoxin spread is thought to depend on the injected amount and the particular neurotoxin preparation. It can result in adverse side effects such as paralysis in nearby muscle tissue, which can largely be avoided by reducing the injected doses to the therapeutically relevant level. Overdosing can also trigger the immune system to generate neutralizing antibodies that inactivate the neurotoxin preventing it from relieving the involuntary muscle activity. Immunologic tolerance to botulinum toxin has been shown to correlate with cumulative doses.

At present, clostridial neurotoxins are still predominantly produced by fermentation processes using appropriate Clostridium strains. However, industrial production of clostridial neurotoxin from anaerobic Clostridium culture is a cumbersome and time-consuming process. Due to the high toxicity of the final product, the procedure must be performed under strict containment. During the fermentation process, the single-chain precursors are proteolytically cleaved by an unknown clostridial protease to obtain the biologically active di-chain clostridial neurotoxin. The degree of neurotoxin activation by proteolytic cleavage varies between different strains and neurotoxin serotypes, which is a major consideration for the manufacture due to the requirement of neurotoxin preparations with a well-defined biological activity. Furthermore, during fermentation processes using Clostridium strains the clostridial neurotoxins are produced as protein complexes, in which the neurotoxic component is embedded by accessory proteins. These accessory proteins have no beneficial effect on biological activity or duration of effect. They can however trigger an immune reaction in the patient, resulting in immunity against the clostridial neurotoxin. Manufacture of recombinant clostridial neurotoxins, which are not embedded by auxiliary proteins, might therefore be advantageous.

Methods for the recombinant expression of clostridial neurotoxins in E. coli are well known in the art (see, for example, WO 00/12728, WO 01/14570, or WO 2006/076902). Furthermore, clostridial neurotoxins have been expressed in eukaryotic expression systems, such as in Pichia pastoris, Pichia methanolica, Saccharomyces cerevisiae, insect cells and mammalian cells (see WO 2006/017749).

Recombinant botulinum neurotoxins may be expressed as single-chain precursors, which subsequently have to be proteolytically cleaved to obtain the final biologically active botulinum neurotoxin. Thus, botulinum neurotoxins may be expressed in high yield in rapidly-growing bacteria as relatively non-toxic single-chain polypeptides.

Furthermore, it might be advantageous to modify botulinum neurotoxin characteristics regarding biological activity, cell specificity, antigenic potential and duration of effect by genetic engineering to obtain recombinant neurotoxins with new therapeutic properties in specific clinical areas. Genetic modification of botulinum neurotoxins might allow altering the mode of action or expanding the range of therapeutic targets.

Botulinum toxin variants exhibiting an increased duration of effect in neuromuscular tissue than naturally occurring botulinum toxins would be very advantageous in order to reduce administration frequency and the incidence of neutralizing antibody generation since immunologic tolerance to botulinum toxin is correlated with cumulative doses.

US 2002/0127247 describes clostridial neurotoxins comprising modifications in secondary modification sites and exhibiting altered biological persistence.

There is a strong demand to produce new botulinum neurotoxins serotype A with an increased duration of effect and with improved properties, in order to allow for exploitation of the therapeutic potential of BoNT serotype A, which have so far been considered impractical for certain clinical application. Ideally, the increased duration of effect of a particular botulinum neurotoxin serotype A could be adjusted in a tailor-made fashion in order to address any particular features and demands of a given indication, such as the amount of neurotoxin being administered, frequency of administration etc. In addition, it would be desirable to produce botulinum neurotoxins serotype A without additional degradation products. To date, such aspects have not been solved satisfactorily.

So far, except for the approach described and claimed in WO 2015/132004, no generally applicable method for modifying clostridial neurotoxins to increase their duration of effect is available. According to WO 2015/132004, a recombinant botulinum neurotoxin comprising a domain consisting of proline (P), alanine (A) and serine (S) residues (hereafter referred to “PASylated” botulinum neurotoxins) exhibits an increased duration of effect compared to a corresponding wildtype botulinum neurotoxin. However, it was shown that by using the protease thrombin for activating such a single-chain precursor neurotoxin serotype A not only the linker was cleaved according to the introduced cleavage sites, but also a further position was cleaved resulting in additional degradation products. Thus, after cleaving the single-chain precursor neurotoxin by using thrombin a mixture of activated BoNT and degradation products was generated. For developing a drug based on a modified BoNT it is desirable that the active ingredient consists of a uniform substance without exhibiting degradation products.

### OBJECTS OF THE INVENTION

It was an object of the invention to overcome the above illustrated drawbacks. In particular, it was an object of the invention to provide a recombinant a single-chain precursor botulinum neurotoxin serotype A which results in a uniform neurotoxin without degradation products after activation with a protease such as thrombin, HRV3C, Tobacco Etch Virus protease, enterokinase or factor Xa and which exhibits an increased duration of effect in comparison to naturally occurring botulinum toxins. It was also an object of the invention to establish a reliable and accurate method for manufacturing and obtaining such a recombinant botulinum neurotoxin. Such a method and novel precursor botulinum neurotoxin used in such methods would serve to satisfy the great need for recombinant botulinum neurotoxins exhibiting an increased duration of effect.

### SUMMARY OF THE INVENTION

The naturally occurring botulinum toxin serotypes display highly divergent durations of effect, probably due to their distinct subcellular localization. BoNT/A exhibits the longest persistence and was shown to localize in the vicinity of the plasma membrane of neuronal cells. However, additional factors such as degradation, spread or diffusion, and/or translocation rates might have a decisive impact on the differences in the duration for the individual botulinum toxin serotypes.

So far, except for the approach described in WO 2015/132004, no generally applicable method for modifying clostridial neurotoxins to increase their duration of effect is available. It was shown that after activating such a single-chain precursor neurotoxin serotype A by using thrombin a mixture consisting of activated BoNT/A and unwanted degradation products was generated.

Surprisingly, it has been found that certain recombinant single-chain precursor botulinum neurotoxins serotype A can be activated by a protease such as thrombin, HRV3C, Tobacco Etch Virus protease, enterokinase or factor Xa without generating unwanted degradation products and these activated neurotoxins exhibit also an increased duration of effect. These single-chain precursor botulinum neurotoxin serotype A can be obtained by a two-fold modification. On the one hand these single-chain precursor neurotoxins comprise at least one additional domain consisting of at least one proline, at least one alanine and at least one serine residues. Secondly, these neurotoxins according to the invention comprise at least one cleavage site for a protease selected from the group consisting of thrombin, HRV3C, Tobacco Etch Virus protease, enterokinase and factor Xa and an amino acid modification located at positions 1273 and/or 1274 within the heavy chain of the neurotoxin according to SEQ ID NO: 1.

Thus, in one aspect, the present invention relates to a recombinant single-chain precursor botulinum neurotoxin serotype A comprising at least one additional domain consisting of at least 50 amino acid residues selected from the group consisting of at least one proline, at least one alanine and at least one serine residues, wherein the single-chain precursor neurotoxin further comprises at least one cleavage site for a protease selected from the group consisting of thrombin, HRV3C, Tobacco Etch Virus protease, enterokinase and factor Xa and an amino acid modification located at positions 1273 and/or 1274 within the heavy chain of the neurotoxin according to SEQ ID NO: 1.

In another aspect, the present invention relates to a recombinant botulinum neurotoxin serotype A obtainable by cleaving the recombinant single-chain precursor neurotoxin according to the invention with a protease selected from the group consisting of thrombin, HRV3C, Tobacco Etch Virus protease, enterokinase and factor Xa.

In another aspect, the present invention relates to a composition, in particular to a pharmaceutical composition comprising the recombinant botulinum neurotoxin of the present invention.

In yet another aspect, the present invention relates to the use of the composition of the present invention for cosmetic treatment.

In another aspect, the present invention relates to a method for the generation of the recombinant botulinum neurotoxin of the present invention.

In another aspect, the present invention relates to a nucleic acid sequence encoding the recombinant single-chain precursor botulinum neurotoxin of the present invention.

In another aspect, the present invention relates to a vector comprising the nucleic acid sequence of the present invention, or the nucleic acid sequence obtainable by the method of the present invention.

In another aspect, the present invention relates to a recombinant host cell comprising the nucleic acid sequence of the present invention, the nucleic acid sequence obtainable by the method of the present invention, or the vector of the present invention.

In another aspect, the present invention relates to a method for producing the recombinant single-chain precursor botulinum neurotoxin of the present invention, comprising the step of expressing the nucleic acid sequence of the present invention, or the nucleic acid sequence obtainable by the method of the present invention, or the vector of the present invention in a recombinant host cell, or cultivating the recombinant host cell of the present invention under conditions that result in the expression of said nucleic acid sequence.

### FIGURES

FIG. 1: Schematic Presentation of a modified botulinum toxin A (PAS-BoNT/A-R1273A-PAS), wherein both the light chain (LC) and the heavy chain (HC) each comprise an additional amino acid sequence consisting of proline (P), alanine (A) and serine (S) residues (PAS) and wherein the heavy chain (HC) comprises an amino acid modification, i.e. the amino acid arginine at position 1273 according to SEQ ID NO: 1 is substituted by an alanine.

FIG. 2: SDS.PAGE of purified DaSch021 (PAS100-BoNT/A-PAS100) and DaSch099 (PAS100-BoNT/A-R1273A-PAS100). Both were treated with thrombin. Lane M: Molecular weight marker. Prior to applying the samples to the gel, f3-mercaptoethanol was added. Lane 1: purified, non-activated single-chain DaSch021 (PAS100-BoNT/A-PAS100) (SC). Light chain (LC) and heavy chain (HC) are shown after activation by 0.4 U thrombin (lane 2) and 4 U thrombin (lane 3) thrombin under reducing conditions. The mutated variant DaSch099 (PAS100-BoNT/A-R1273A-PAS100) is shown in lane 4 (single-chain) and after activation by 0.4 U thrombin (lane 5) and 4 U thrombin (lane 6). Treatment of DaSch021 with thrombin leads to an unwanted cleavage of the heavy chain and the generation of a secondary cleavage product (*). Added thrombin is marked as Thr.

FIG. 3: Mouse running assay with PAS100-BoNT/A-R1273A-PAS100 Equipotent dosages of PAS100-BoNT/A-R1273A-PAS100 (Dasch099 (9pg), curve (1)) were injected into the M. gastrocnemius of each mice in comparison to standard Xeomin® (0.6U;3pg, see “Std. 81208”, curve (2) and mean values of 17 assays of “Std. 81208”, curve (4)) and 9pg of PASylated Botulinum Toxin Type A without the introduced mutation (=Dasch021, curve (5)). The mice had been trained in a treadmill. The daily running distance in the treadmill was measured over 21 days. The paralysis caused by the toxins was plotted as percentage of the running distance on the day before the injection, which was set as 100%, against the time.

### DETAILED DESCRIPTION OF THE INVENTION

The present invention may be understood more readily by reference to the following detailed description of the invention and the examples included therein.

In one aspect, the present invention relates to a recombinant single-chain precursor botulinum neurotoxin serotype A comprising at least one additional domain consisting of at least 50 amino acid residues selected from the group consisting of at least one proline, at least one alanine and at least one serine residues, wherein the single-chain precursor neurotoxin further comprises at least one cleavage site for a protease selected from the group consisting of thrombin, HRV3C, Tobacco Etch Virus protease, enterokinase and factor Xa and an amino acid modification located at position 1273 and/or at position 1274 within the heavy chain of the neurotoxin according to SEQ ID NO: 1.

In particular embodiments, said additional domain comprises a plurality of amino acid repeats, wherein said repeat consist of proline, alanine and serine residues and wherein no more than six consecutive amino acid residues are identical.

In particular embodiments, the proline residues comprised in said additional domain constitute more than 4% and less than 40% of the amino acids of said domain.

In particular embodiments, said additional domain comprises at least one amino acid sequence selected from the group consisting of: ASPAAPAPASPAAPAPSAPA; AAPASPAPAAPSAPAPAAPS; APSSPSPSAPSSPSPASPSS, SAPSSPSPSAPSSPSPASPS, SSPSAPSPSSPASPSPSSPA, AASPAAPSAPPAAASPAAPSAPPA, and ASAAAPAAASAAASAPSAAA or circular permuted versions or (a) multimers(s) of these sequences as a whole or parts of these sequences, particularly (ASPAAPAPASPAAPAPSAPA)n, with n being an integer selected from 3 to 25, more particularly from 4 to 8, more particularly from 5 to 10, in particular wherein n is 5 or 10.

In the context of the present invention, the term “single-chain precursor botulinum neurotoxin” refers to a single-chain precursor for a disulfide-linked di-chain botulinum neurotoxin serotype A, comprising a functionally active botulinum neurotoxin light chain, a functionally active neurotoxin heavy chain, and a loop region linking the C-terminus of the light chain with the N-terminus of the heavy chain.

In the context of the present invention, the term “recombinant single-chain precursor botulinum neurotoxin” refers to a single-chain precursor botulinum neurotoxin serotype A, comprising at least one heterologous domain, i.e. a domain independently selected from a species other than Clostridium botulinum.

In particular embodiments, the recombinant single-chain precursor botulinum neurotoxin comprises a cleavage site for a protease selected from the group consisting of thrombin, HRV3C, Tobacco Etch Virus protease, enterokinase and factor Xa in said loop region.

Single-chain precursor botulinum neurotoxins have to be proteolytically cleaved to obtain the final biologically active botulinum neurotoxins. Proteolytic cleavage may either occur during heterologous expression by host cell enzymes, or by adding proteolytic enzymes to the raw protein material isolated after heterologous expression. Naturally occurring botulinum neurotoxins usually contain one or more cleavage signals for proteases which post-translationally cleave the single-chain precursor molecule, so that the final di- or multimeric complex can form. At present, botulinum neurotoxins are still predominantly produced by fermentation processes using appropriate Clostridium strains. During the fermentation process, the single-chain precursors are proteolytically cleaved by an unknown clostridial protease to obtain the biologically active di-chain clostridial neurotoxin. In cases, where the single-chain precursor molecule is the precursor of a protease, autocatalytic cleavage may occur. Alternatively, the protease can be a separate non-clostridial enzyme expressed in the same cell. WO 2006/076902 describes the proteolytic cleavage of a recombinant clostridial neurotoxin single-chain precursor at a heterologous recognition and cleavage site by incubation of the E. coli host cell lysate. The proteolytic cleavage is carried out by an unknown E. coli protease. In certain applications of recombinant expression, modified protease cleavage sites have been introduced recombinantly into the interchain region between the light and heavy chain of clostridial toxins, e.g. protease cleavage sites for human thrombin or non-human proteases (see WO 01/14570).

In a particular embodiment, the recombinant single-chain precursor botulinum neurotoxin further comprises a binding tag, particularly selected from the group comprising: glutathione-S-transferase (GST), maltose binding protein (MBP), a His-tag, a StrepTag, or a FLAG-tag.

In the context of the present invention, the term “botulinum neurotoxin” refers to a natural neurotoxin obtainable from bacteria Clostridium botulinum, or to a neurotoxin obtainable from alternative sources, including from recombinant technologies or from genetic or chemical modification. Particularly, the botulinum neurotoxins have endopeptidase activity.

Botulinum neurotoxins are produced as single-chain precursors that are proteolytically cleaved by an unknown clostridial endoprotease within the loop region to obtain the biologically active disulfide-linked di-chain form of the neurotoxin, which comprises two chain elements, a functionally active light chain and a functionally active heavy chain, where one end of the light chain is linked to one end of the heavy chain not via a peptide bond, but via a disulfide bond.

In the context of the present invention, the term “botulinum neurotoxin light chain” refers to that part of a botulinum neurotoxin that comprises an endopeptidase activity responsible for cleaving one or more proteins that is/are part of the so-called SNARE-complex involved in the process resulting in the release of neurotransmitter into the synaptic cleft: In naturally occurring botulinum neurotoxins, the light chain has a molecular weight of approx. 50 kDa.

In the context of the present invention, the term “botulinum neurotoxin heavy chain” refers to that part of a botulinum neurotoxin that is responsible for entry of the neurotoxin into the neuronal cell: In naturally occurring botulinum neurotoxins, the heavy chain has a molecular weight of approx. 100 kDa.

In the context of the present invention, the term “functionally active botulinum neurotoxin chain” refers to a recombinant clostridial neurotoxin light/heavy chain able to perform the biological functions of a naturally occurring Clostridium botulinum neurotoxin chain to at least about 50%, particularly to at least about 60%, to at least about 70%, to at least about 80%, and most particularly to at least about 90%, where the biological functions of botulinum neurotoxin chains include, but are not limited to, binding of the heavy chain to the neuronal cell, entry of the neurotoxin into a neuronal cell, release of the light chain from the di-chain neurotoxin, and endopeptidase activity of the light chain. Methods for determining a neurotoxic activity can be found, for example, in WO 95/32738, which describes the reconstitution of separately obtained light and heavy chains of tetanus toxin and botulinum toxin. Also cell-based assay methods as described for example in WO2009/114748, WO 2013/049508 and WO2014/207109.

In the context of the present invention, the term “about” or “approximately” means within 20%, alternatively within 10%, including within 5% of a given value or range. Alternatively, especially in biological systems, the term “about” means within about a log (i.e. an order of magnitude), including within a factor of two of a given value.

In the context of the present invention, the term “recombinant botulinum neurotoxin” refers to a composition comprising a botulinum neurotoxin that is obtained by expression of the neurotoxin in a heterologous cell such as E. coli, and including, but not limited to, the raw material obtained from a fermentation process (supernatant, composition after cell lysis), a fraction comprising a botulinum neurotoxin obtained from separating the ingredients of such a raw material in a purification process, an isolated and essentially pure protein, and a formulation for pharmaceutical and/or aesthetic use comprising a botulinum neurotoxin and additionally pharmaceutically acceptable solvents and/or excipients.

In the context of the present invention, the term “comprises” or “comprising” means “including, but not limited to”. The term is intended to be open-ended, to specify the presence of any stated features, elements, integers, steps or components, but not to preclude the presence or addition of one or more other features, elements, integers, steps, components, or groups thereof. The term “comprising” thus includes the more restrictive terms “consisting of” and “consisting essentially of”.

In particular embodiments, the recombinant single-chain precursor botulinum neurotoxin serotype A according to the invention comprises at least one domain comprising an amino acid sequence consisting of between 50 and 500 amino acid residues, more particularly between 70 and 300 amino acid residues, more particularly between 80 and 220 amino acid residues, particularly 100 amino acid residues, 150 amino acid residues, or 200 amino acid residues.

In particular embodiments, the recombinant single-chain precursor botulinum neurotoxin serotype A according to the invention comprises at least one domain, wherein said at least one domain is inserted at a position selected from (i) the N-terminus of the light chain of said recombinant neurotoxin; (ii) the C-terminus of the light chain of said recombinant neurotoxin; (iii) the N-terminus of the heavy chain of said recombinant neurotoxin; or (iv) the C-terminus of the heavy chain of said recombinant neurotoxin.

In particular embodiments, the recombinant single-chain precursor botulinum neurotoxin serotype A according to the invention comprises an amino acid modification wherein the amino acid arginine at position 1273 according to SEQ ID NO: 1 is substituted by an amino acid selected from the group consisting of alanine, methionine, glutamine, leucine, isoleucine, phenylalanine, threonine, valine, tyrosine and serine.

In particular embodiments, the recombinant single-chain precursor botulinum neurotoxin serotype A according to the invention comprises an amino acid modification wherein the amino acid serine at position 1274 according to SEQ ID NO: 1 is substituted by an amino acid selected from the group consisting of aspartic acid, tyrosine, asparagine, glutamic acid.

In particular embodiments, the recombinant single-chain precursor botulinum neurotoxin serotype A according to the invention comprises an amino acid modification wherein the amino acids at positions 1273 and 1274 according to SEQ ID NO: 1 are substituted, wherein position 1273 is substituted by an amino acid selected from the group consisting of alanine, methionine, glutamine, leucine, isoleucine, phenylalanine, threonine, valine, tyrosine and serine and position 1274 is substituted by an amino acid selected from the group consisting of aspartic acid, tyrosine, asparagine, glutamic acid.

In particular embodiments, the recombinant single-chain precursor botulinum neurotoxin serotype A according to the invention comprises two domains consisting of at least one proline, at least one alanine and at least one serine residues.

In particular embodiments, the recombinant single-chain precursor botulinum neurotoxin serotype A according to the invention comprises two domains consisting of at least one proline, at least one alanine and at least one serine residues, wherein one domain is inserted at the N-terminus of the light chain of said recombinant neurotoxin and one domain is inserted at the C-terminus of the heavy chain of said recombinant neurotoxin.

In the context of the present invention, the term “functional variant of a botulinum neurotoxin” refers to a neurotoxin that differs in the amino acid sequence and/or the nucleic acid sequence encoding the amino acid sequence from a botulinum neurotoxin, but is still functionally active. In the context of the present invention, the term “functionally active” refers to the property of a recombinant botulinum neurotoxin to exhibit a biological activity of at least about 20%, particularly to at least about 40%, at least about 70%, at least about 80%, and most particularly at least about 90% of the biological activity of a naturally occurring parental botulinum neurotoxin, i.e. a parental botulinum neurotoxin without modifications at the C-terminus of the light chain, where the biological functions include, but are not limited to, binding to the neurotoxin receptor, entry of the neurotoxin into a neuronal cell, release of the light chain from the two-chain neurotoxin, and endopeptidase activity of the light chain, and thus inhibition of neurotransmitter release from the affected nerve cell. In vivo assays for assessing biological activity include the mouse LD50 assay and the ex vivo mouse hemidiaphragm assay as described by Pearce et al. (Pearce 1994, Toxicol. Appl. Pharmacol. 128: 69-77) and Dressler et al. (Dressler 2005, Mov. Disord. 20:1617-1619, Keller 2006, Neuroscience 139: 629-637) or a cell-based assay as described in WO2009/114748, WO2014/207109 or WO 2013/049508. The biological activity is commonly expressed in Mouse Units (MU). As used herein, 1 MU is the amount of neurotoxic component, which kills 50% of a specified mouse population after intraperitoneal injection, i.e. the mouse i.p. LD50.

On the protein level, a functional variant will maintain key features of the corresponding botulinum neurotoxin serotype A, such as key residues for the endopeptidase activity in the light chain, or key residues for the attachment to the neurotoxin receptors or for translocation through the endosomal membrane in the heavy chain, but may contain modifications comprising a substitution of one or more amino acids of the corresponding botulinum neurotoxin.

In another embodiment, the functional variant of a botulinum neurotoxin additionally comprises a signal peptide. Usually, said signal peptide will be located at the N-terminus of the neurotoxin. Many such signal peptides are known in the art and are comprised by the present invention. In particular, the signal peptide results in transport of the neurotoxin across a biological membrane, such as the membrane of the endoplasmic reticulum, the Golgi membrane or the plasma membrane of a eukaryotic or prokaryotic cell. It has been found that signal peptides, when attached to the neurotoxin, will mediate secretion of the neurotoxin into the supernatant of the cells. In certain embodiments, the signal peptide will be cleaved off in the course of, or subsequent to, secretion, so that the secreted protein lacks the N-terminal signal peptide, is composed of separate light and heavy chains, which are covalently linked by disulfide bridges, and is proteolytically active.

In particular embodiments, the functional variant has a sequence identity of at least about 40%, at least about 50%, at least about 60%, at least about 70% or most particularly at least about 80%, and a sequence homology of at least about 60%, at least about 70%, at least about 80%, at least about 90%, or most particularly at least about 95% to the corresponding part in the parental botulinum neurotoxin serotype A. Methods and algorithms for determining sequence identity and/or homology, including the comparison of variants having deletions, additions, and/or substitutions relative to a parental sequence, are well known to the practitioner of ordinary skill in the art. The term “identity” as used herein refers to sequence identity characterized by determining the number of identical amino acids between two nucleic acid sequences or two amino acid sequences wherein the sequences are aligned so that the highest order match is obtained. It can be calculated using published techniques or methods codified in computer programs such as, for example, BLASTP, BLASTN or FASTA (Altschul 1990, J Mol Biol 215, 403). The percent identity values are, in one aspect, calculated over the entire amino acid sequence. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (Higgins 1989, CABIOS 5, 151) or the programs Gap and BestFit (Needleman 1970, J Mol Biol 48; 443; Smith 1981, Adv Appl Math 2, 482), which are part of the GCG software packet (Genetics Computer Group 1991, 575 Science Drive, Madison, Wis., USA 53711), may be used. The sequence identity values recited above in percent (%) are to be determined, in another aspect of the invention, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments. On the DNA level, the nucleic acid sequences encoding the functional homologue and the parental botulinum neurotoxin may differ to a larger extent due to the degeneracy of the genetic code. It is known that the usage of codons is different between prokaryotic and eukaryotic organisms. Thus, when expressing a prokaryotic protein such as a botulinum neurotoxin, in a eukaryotic expression system, it may be necessary, or at least helpful, to adapt the nucleic acid sequence to the codon usage of the expression host cell, meaning that sequence identity or homology may be rather low on the nucleic acid level.

In the context of the present invention, the term “variant” refers to a botulinum neurotoxin that is a chemically, enzymatically, or genetically modified derivative of a corresponding neurotoxin of C. botulinum neurotoxin serotype A. A chemically modified derivative may be one that is modified by pyruvation, phosphorylation, sulfatation, lipidation, pegylation, glycosylation and/or the chemical addition of an amino acid or a polypeptide comprising between 2 and about 100 amino acids, including modification occurring in the eukaryotic host cell used for expressing the derivative. An enzymatically modified derivative is one that is modified by the activity of enzymes, such as endo- or exoproteolytic enzymes, including modification by enzymes of the eukaryotic host cell used for expressing the derivative. As pointed out above, a genetically modified derivative is one that has been modified by deletion or substitution of one or more amino acids contained in, or by addition of one or more amino acids (including polypeptides comprising between 2 and about 100 amino acids) to, the amino acid sequence of said botulinum neurotoxin. Methods for designing and constructing such chemically or genetically modified derivatives and for testing of such variants for functionality are well known to anyone of ordinary skill in the art.

In another embodiment, the recombinant botulinum neurotoxin serotype A according to the invention is obtainable by cleaving the recombinant single-chain precursor neurotoxin according to the invention with a protease selected from the group consisting of thrombin, HRV3C, Tobacco Etch Virus protease, enterokinase and factor Xa.

In particular embodiments, the recombinant botulinum neurotoxin serotype A according to the invention shows an increased duration of effect relative to a wildtype botulinum neurotoxin serotype A.

In particular embodiments, the recombinant botulinum neurotoxin serotype A according to the invention is used in the treatment of a disease requiring improved chemodenervation, wherein the recombinant neurotoxin causes an increased duration of effect relative to a wildtype botulinum neurotoxin serotype A.

In the context of the present invention, the term “increased duration of effect” or “increased duration of action” refers to a longer lasting denervation mediated by a clostridial neurotoxin of the present invention. For example, as disclosed herein, administration of a disulfide-linked di-chain clostridial neurotoxin comprising at least one domain according to the invention results in localized paralysis for a longer period of time relative to administration of an identical disulfide-linked di-chain clostridial neurotoxin without the at least one domain according to the present invention.

In the context of the present invention, the term “increased duration of effect/action” is defined as a more than about 20%, particularly more than about 50%, more particularly more than about 90% increased duration of effect of the recombinant neurotoxin of the present invention relative to the identical neurotoxin without the at least one domain according to the invention. For example, an “increased duration of effect” can be determined using the “Mouse Running Assay”. The “Mouse Running Assay” is well-known to the person skilled in the art and measures the daily running distance of a mouse in a treadmill after a botulinum neurotoxin was injected into the M. gastrocnemius (see Keller J E. Recovery from botulinum neurotoxin poisoning in vivo. Neuroscience. 2006 May 12;139(2):629-37). The distance which a mouse is able to run in the treadmill the day before the botulinum neurotoxin is injected is used as comparison and is set as 100%. A daily running distance of no more than 80% of the initial running distance is regarded as paralysis of the muscle. The duration of effect is determined by the time period between the time point attaining a half-maximal paralysis and the time point when paralysis reaches recovery, i.e. 40% of the initial running distance. If this time period is longer than 2 days compared with the standard (wildtype BoNT), the botulinum neurotoxin is considered to exhibit an “increased duration of effect/action” provided that the mutated BoNT exhibits a similar potency i.e shows a similar maximal paralysis (reduction of the running distance) of about 80-90%.

In the context of the present invention the term “denervation” refers to denervation resulting from administration of a chemodenervating agent, for example a neurotoxin.

In the context of the present invention, the term “localized denervation” or “localized paralysis” refers to denervation of a particular anatomical region, usually a muscle or a group of anatomically and/or physiologically related muscles, which results from administration of a chemodenervating agent, for example a neurotoxin, to the particular anatomical region.

Without wishing to be bound by theory, the recombinant botulinum neurotoxins of the present invention might show increased biological half-life, reduced degradation rates, decreased diffusion rates, increased uptake by neuronal cells, and/or modified intracellular translocation rates, in each case relative to an identical parental clostridial neurotoxin without the at least two domains according to the invention.

In the context of the present invention, the term “biological half-life” specifies the lifespan of a protein, for example of a botulinum neurotoxin serotype A, in vivo. In the context of the present invention, the term “biological half-life” refers to the period of time, by which half of a protein pool is degraded in vivo. For example it refers to the period of time, by which half of the amount of botulinum neurotoxin of one administered dosage is degraded.

In another aspect, the present invention relates to a composition, in particular a pharmaceutical or cosmetic composition comprising the recombinant botulinum neurotoxin of the present invention. For preparing a preparation comprising a botulinum neurotoxin the toxin can be formulated by various techniques dependent on the desired application purposes which are known in the art. For example, the (biologically active) botulinum neurotoxin polypeptide can be used in combination with one or more pharmaceutically acceptable carriers as a pharmaceutical composition. The pharmaceutically acceptable carrier(s) must be acceptable in the sense of being compatible with the other ingredients of the formulation and being not deleterious to the recipient thereof. The pharmaceutical carrier employed may include a solid, a gel, or a liquid. Exemplary of solid carriers are lactose, terra alba, sucrose, talc, gelatine, agar, pectin, acacia, magnesium stearate, stearic acid and the like. Exemplary of liquid carriers are glycerol, phosphate buffered saline solution, water, emulsions, various types of wetting agents, and the like. Suitable carriers comprise those mentioned above and others well known in the art, see, e.g., Remington's Pharmaceutical Sciences, Mack Publishing Company, Easton, Pa. In an aspect, the pharmaceutical composition can be dissolved in a diluent, prior to administration. The diluent is also selected so as not to affect the biological activity of the Neurotoxin product. Examples of such diluents are distilled water or physiological saline. In addition, the pharmaceutical composition or formulation may also include other carriers or non-toxic, non-therapeutic, non-immunogenic stabilizers and the like. Thus, the formulated Neurotoxin product can be present, in an aspect, in liquid or lyophilized form. In an aspect, it can be present together with glycerol, protein stabilizers (HSA) or non-protein stabilizers such as polyvinyl pyrrolidone (PVP), hyaluronic acid or free amino acids. In an aspect, suitable non-proteinaceous stabilizers are disclosed in WO 2005/007185 or WO 2006/020208. The formulated Neurotoxin product may be used for human or animal therapy of various diseases or disorders in a therapeutically effective dose or for cosmetic purposes.

In particular embodiments, the recombinant botulinum neurotoxin of the present invention or the pharmaceutical composition of the present invention is for use in the treatment of a disease or condition taken from the list of: cervical dystonia (spasmodic torticollis), blepharospasm, severe primary axillary hyperhidrosis, achalasia, lower back pain, benign prostate hypertrophy, chronic focal painful neuropathies, migraine and other headache disorders.

Additional indications where treatment with botulinum neurotoxins is currently under investigation and where the pharmaceutical composition of the present invention may be used, include pediatric incontinence, incontinence due to overactive bladder, and incontinence due to neurogenic bladder, anal fissure, spastic disorders associated with injury or disease of the central nervous system including trauma, stroke, multiple sclerosis, Parkinson's disease, or cerebral palsy, focal dystonias affecting the limbs, face, jaw or vocal cords, temporomandibular joint (TMJ) pain disorders, diabetic neuropathy, wound healing, excessive salivation, vocal cord dysfunction, reduction of the Masseter muscle for decreasing the size of the lower jaw, treatment and prevention of chronic headache and chronic musculoskeletal pain, treatment of snoring noise, assistance in weight loss by increasing the gastric emptying time.

Most recently, clostridial neurotoxins have been evaluated for the treatment of other new indications, for example painful keloid, diabetic neuropathic pain, refractory knee pain, trigeminal neuralgia trigger-zone application to control pain, scarring after cleft-lip surgery, cancer and depression.

In yet another aspect, the present invention relates to the use of the composition of the present invention for cosmetic treatment.

Thus, in another aspect, the present invention relates to a method of cosmetically treating a patient, comprising the step of administering a composition comprising a recombinant clostridial neurotoxin according to the present invention to a patient desiring such cosmetic treatment.

In the context of the present invention, the term “cosmetic treatment” relates to uses in cosmetic or aesthetic applications, such as the treatment of wrinkles, crow's feet, glabella frown lines, reduction of the masseter muscle, reduction of the calves, removing of facial asymmetries etc.

In another aspect, the present invention relates to a method for the generation of the recombinant botulinum neurotoxin of the present invention, comprising the steps of:
obtaining a recombinant nucleic acid sequence encoding a recombinant single-chain precursor neurotoxin by the insertion of at least one nucleic acid sequence encoding said PAS-domain into a nucleic acid sequence encoding a parental neurotoxin and
by modifying the nucleic acid sequence encoding a botulinum neurotoxin at position 1273 and/or 1274 of the heavy chain of the neurotoxin according to SEQ ID NO: 1,
by inserting at least one cleavage site for a protease selected from the group consisting of thrombin, HRV3C, Tobacco Etch Virus protease, enterokinase and factor Xa in the loop region,
by heterologously expressing said recombinant nucleic acid sequence in a host cell, particularly in a bacterial host cell, more particularly in an E. coli host cell,
by cleaving the recombinant single-chain precursor neurotoxin with a protease selected from the group consisting of thrombin, HRV3C, Tobacco Etch Virus protease, enterokinase and factor Xa.

In the context of the present invention, the term “recombinant nucleic acid sequence” refers to a nucleic acid, which has been generated by joining genetic material from two different sources.

In the context of the present invention, the term “parental botulinum neurotoxin” refers to an initial botulinum neurotoxin without modifications selected from a natural botulinum neurotoxin, a functional variant of a natural botulinum neurotoxin or a chimeric botulinum neurotoxin.

In certain embodiments, the E. coli cells are selected from E. coli XL1-Blue, Nova Blue, TOP10, XL10-Gold, BL21, and K12.

In particular embodiments, the method for the generation of the recombinant botulinum neurotoxin of the present invention additionally comprises at least one of the steps of (i) generating a disulfide-linked di-chain recombinant botulinum neurotoxin according to the invention by causing or allowing contacting of said recombinant single-chain precursor botulinum neurotoxin with an endoprotease and (ii) purification of said recombinant single-chain precursor botulinum neurotoxin or said disulfide-linked di-chain recombinant botulinum neurotoxin by chromatography.

In particular embodiments, the recombinant single-chain precursor botulinum neurotoxin, or the recombinant disulfide-linked di-chain botulinum neurotoxin, is purified after expression, or in the case of the recombinant disulfide-linked di-chain botulinum neurotoxin, after the cleavage reaction. In particular such embodiments, the protein is purified by chromatography, particularly by immunoaffinity chromatography, or by chromatography on an ion exchange matrix, a hydrophobic interaction matrix, or a multimodal chromatography matrix, particularly a strong ion exchange matrix, more particularly a strong cation exchange matrix.

In the context of the present invention, the term “causing . . . contacting of said recombinant single-chain precursor botulinum neurotoxin . . . with an endoprotease” refers to an active and/or direct step of bringing said neurotoxin and said endoprotease in contact, whereas the term “allowing contacting of a recombinant single-chain precursor botulinum neurotoxin . . . with an endoprotease” refers to an indirect step of establishing conditions in such a way that said neurotoxin and said endoprotease are getting in contact to each other.

In the context of the present invention, the term “endoprotease” refers to a protease that breaks peptide bonds of non-terminal amino acids (i.e. within the polypeptide chain). As they do not attack terminal amino acids, endoproteases cannot break down peptides into monomers.

In particular embodiments, cleavage of the recombinant single-chain precursor botulinum neurotoxin is near-complete.

In the context of the present invention, the term “near-complete” is defined as more than about 95% cleavage, particularly more than about 97.5%, more particularly more than about 99% as determined by SDS-PAGE and subsequent Western Blot or reversed phase chromatography.

In particular embodiments, cleavage of the recombinant single-chain precursor botulinum neurotoxin occurs at a heterologous cleavage signal located in the loop region of the recombinant precursor botulinum neurotoxin.

In particular embodiments, the cleavage reaction is performed with crude host cell lysates containing said single-chain precursor protein.

In other particular embodiments, the single-chain precursor protein is purified or partially purified, particularly by a first chromatographic enrichment step, prior to the cleavage reaction.

In the context of the present invention, the term “purified” relates to more than about 90% purity. In the context of the present invention, the term “partially purified” relates to purity of less than about 90% and an enrichment of more than about two fold.

In another aspect, the present invention relates to a method for obtaining the nucleic acid sequence of the present invention, comprising the step of modifying a nucleic acid sequence encoding a parental botulinum neurotoxin.

In another aspect, the present invention relates to a vector comprising the nucleic acid sequence of the present invention, or the nucleic acid sequence obtainable by the method of the present invention.

In another aspect, the present invention relates to a recombinant host cell comprising the nucleic acid sequence of the present invention, the nucleic acid sequence obtainable by the method of the present invention, or the vector of the present invention.

In another aspect, the present invention relates to a method for producing the recombinant single-chain precursor botulinum neurotoxin of the present invention, comprising the step of expressing the nucleic acid sequence of the present invention, or the nucleic acid sequence obtainable by the method of the present invention, or the vector of the present invention in a recombinant host cell, or cultivating the recombinant host cell of the present invention under conditions that result in the expression of said nucleic acid sequence.

### EXAMPLES

### Example 1

### Generation and Purification of a PASylated Botulinum Toxin Type A (PAS100-BoNT/A-R1273A-PAS100)

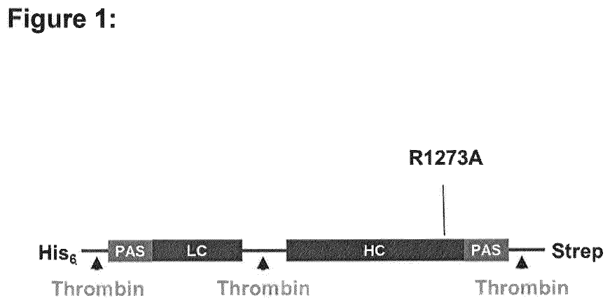

The nucleic acid construct encoding a “PAS” module comprising 100 amino acid residues built from the amino acids proline (P), alanine (A) and serine (S) was synthetically produced, wherein the following motive was used (ASPAAPAPASPAAPAPSAPA)₅. A BoNT/A nucleic acid construct including a “PAS” module at the N-terminus and the C-terminus and further including a thrombin cleavage site in the linker region and for splitting-off tags was produced. The mutation R1273A was inserted via QuickChange™. The sequences were synthetically produced. By using restriction enzymes Ndel and Swal, the corresponding gene module was first inserted at the N-terminus of recombinant BoNT/A (rBoNT/A). In a second step, the PAS module was inserted at the C-terminus of the heavy chain by using restriction enzymes Bglll and Aatll. The correct cloning was verified by sequencing. For expression of BoNT/A in E.coli gene constructs were cloned into pET29c. The variants contain fused His6- and Strep-affinity tags which can be cleaved after protein purification via thrombin (see FIG. 1).

### Protein Expression and Purification.

Expression of rBoNT/A variants was performed in Riesenberg media with 50 μg/mL Kanamycin{Riesenberg, 1991 #1}. Cells were grown in shake flasks (37° C., 175 r.p.m) until an OD600 of 1.5-2 was reached. For induction of protein expression 1 mM IPTG (Fermentas) was added to the E.coli culture. Protein synthesis was performed for 24 h (15 ° C., 175 r.p.m.). Cells were collected by centrifugation (5,000 r.p.m., 20 min, 4° C.) and resuspended in His binding buffer pH 8.0 (50 mM Tris, 150 mM NaCl, 5 mM Imidazol) containing EDTA-free protease inhibitor complete (Roche Diagnostics). For the determination of endopeptidase activity and in vivo characterization, the different toxin variants were extracted and purified. Resuspended pellets were disrupted in 2-3 cycles by a French Press Cell Disrupter (Thermo Electron Corporation) at 4° C. The resulting crude extracts were centrifuged (20,000 r.p.m., 30 min, 4° C.), and the supernatants with the soluble proteins were recovered. Protein purification was carried out by fast protein liquid chromatography (GE Healthcare) using a three step purification protocols. The first capture step was performed by IMAC using a HisTrap HP 1 mL column (GE Healthcare). The column was washed (1 ml min-1 working flow) using a two-step protocol with His elution buffer (50 mM Tris, 150 mM NaCl, 400 mM Imidazol pH 8,0). The elution of the toxin proteins occurred at 400 mM Imidazol. In a further step a Strep-Tactin affinity chromatography was performed as previously described (IBA GmbH). As an alternative instead of a second affinity chromatography a cation exchange chromatography with a HiTrap SP HP 1 mL column (GE Healthcare, Freiburg, Germany) was used. The corresponding samples were diluted with SP binding buffer (50 mM Tris, pH 8) and eluted with SP elution buffer (50 mM Tris, 1 M NaCl, pH 8). This procedure was followed by a SEC using a Superdex 200 10/300 column (GE Healthcare). The SEC running buffer (20 mM Tris, 150 mM NaCl, 2,5 mM CaCl2 pH 7,7) was also used to store the purified protein solutions in aliquots at −20° C. Each protein was analyzed by applying 0,5-1 μg on 4-12% gradient SDS-PAGE (Novex Life Technologies) and stained with Coomassie G-250 based SimplyBlue safe stain (Pierce). Each protein was judged >98% pure before applying in vitro or in vivo experiments.

Thrombin Cleavage

BoNT/A preparations were activated with Thrombin (Merck Millipore; 8 U/1 mg BoNT) for 24 h at 20° C. yielding >99% of di-chain toxin. Afterwards the cleavage protease was eliminated with the previously described Strep-Tactin Kit (IBA GmbH).

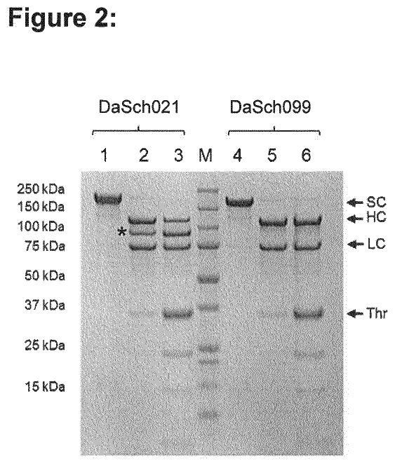

Expression was performed in expression strain E. coli BI21. Purification was done using a combination of His affinity, ion exchange and size exclusion chromatography, followed by activation using thrombin. FIG. 2 summarizes the results of purification and activation.

### Example 2

### Duration of Effect of PAS100-BoNT/A-R1273A-PAS100 in the “Mouse Running Assay”

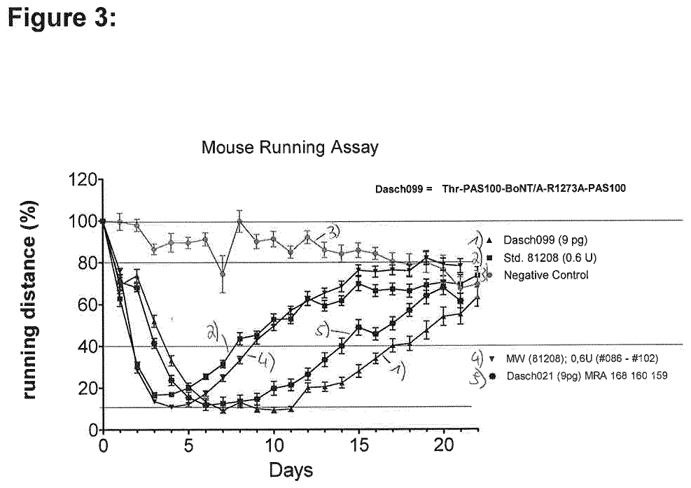

Equipotent dosages of PAS100-BoNT/A-R1273A-PAS100 (Dasch099 (9pg) were injected into the M. gastrocnemius of each mice in comparison to standard Xeomin® (0.6U;3pg, see “Std. 81208”, curve (2), FIGS. 3) and 9pg of PASylated Botulinum Toxin Type A without the introduced mutation (=Dasch021). The mice had been trained in a treadmill. The daily running distance in the treadmill was measured over 21 days. The paralysis caused by the toxins was plotted as percentage of the running distance on the day before the injection, which was set as 100%, against the time (see FIG. 3).

As shown in FIG. 3, the injection of PAS100-BoNT/A-R1273A-PAS100, resulted in an increased duration of effect compared to standard Xeomin®. During the recovery phase the running distance of the control group (mean of standard (17 assays) from Xeomin®, see curve (4)) reached a value of 40% of the starting value 7 days after half-maximum paralysis was observed (day 9), whereas the group treated with PAS100-BoNT/A-R1273A-PAS100 reached that value 15 days after half-maximum paralysis (day 17). Thus, the duration of effective paralysis was significantly extended.

TABLE 1
Sequences

SEQ ID NO 1: recombinant BoNT A including His.tag (amino
acid sequence)
SEQ ID NO 2: recombinant single-chain precursor PAS100-BoNT/
A-R1273A-PAS100 including His.tag (amino acid sequence)
SEQ ID NO 3: recombinant single-chain precursor PAS100-BoNT/
A-R1273A-PAS100 including His.tag (nucleic acid sequence)
SEQ ID NO 1:
mqfvnkqfny kdpvngvdia yikipnvgqm qpvkafkihn kiwviperdt ftnpeegdln
pppeakqvpv syydstylst dnekdnylkg vtklferiys tdlgrmllts ivrgipfwgg
stidtelkvi dtncinviqp dgsyrseeln lviigpsadi iqfecksfgh evlnltrngy
gstqyirfsp dftfgfeesl evdtnpllga gkfatdpavt lahelihagh rlygiainpn
rvfkvntnay yemsglevsf eelrtfgghd akfidslqen efrlyyynkf kdiastlnka
ksivgttasl qymknvfkek yllsedtsgk fsvdklkfdk lykmlteiyt ednfvkffkv
lnrktylnfd kavfkinivp kvnytiydgf nlrntnlaan fngqnteinn mnftklknft
glfefykllc vrgiitsktk sldkgynkal ndlcikvnnw dlffspsedn ftndlnkgee
itsdtnieaa eenisldliq qyyltfnfdn epenisienl ssdiigqlel mpnierfpng
kkyeldkytm fhylraqefe hgksrialtn svneallnps rvytffssdy vkkvnkatea
amflgwveql vydftdetse vsttdkiadi tiiipyigpa lnignmlykd dfvgalifsg
avillefipe iaipvlgtfa lvsyiankvl tvqtidnals krnekwdevy kyivtnwlak
vntqidlirk kmkealenqa eatkaiinyq ynqyteeekn ninfniddls sklnesinka
mininkflnq csysylmnsm ipygykrled fdaslkdall kyiydnrgtl igqvdrlkdk
vnntlstdip fqlskyvdnq rllstfteyi kniintsiln lryesnhlid lsryaskini
gskvnfdpid knqiqlfnle sskievilkn aivynsmyen fstsfwirip kyfnsislnn
eytiincmen nsgwkvslny geiiwtlqdt qeikqrvvfk ysqminisdy inrwifvtit
nnrlnnskiy ingrlidqkp isnlgnihas nnimfkldgc rdthryiwik yfnlfdkeln
ekeikdlydn qsnsgilkdf wgdylqydkp yymlnlydpn kyvdvnnvgi rgymylkgpr
gsvmttniyl nsslyrgtkf iikkyasgnk dnivrnndrv yinvvvknke yrlatnasqa
gvekilsale ipdvgnlsqv vvmkskndqg itnkckmnlq dnngndigfi gfhqfnniak
lvasnwynrq ierssrtlgc swefipvddg wgerpl
SEQ ID NO 2: recombinant single-chain precursor PAS100-BoNT/
A-R1273A-PAS100 including His.tag (amino acid sequence)
MGSSHHHHHH GSLVPRSSSA SPAAPAPASP AAPAPSAPAA SPAAPAPASP AAPAPSAPAA
SPAAPAPASP AAPAPSAPAA SPAAPAPASP AAPAPSAPAA SPAAPAPASP AAPAPSAPAA
PFVNKQFNYK DPVNGVDIAY IKIPNAGQMQ PVKAFKIHNK IWVIPERDTF TNPEEGDLNP
PPEAKQVPVS YYDSTYLSTD NEKDNYLKGV TKLFERIYST DLGRMLLTSI VRGIPFWGGS
TIDTELKVID TNCINVIQPD GSYRSEELNL VIIGPSADII QFECKSFGHE VLNLTRNGYG
STQYIRFSPD FTFGFEESLE VDTNPLLGAG KFATDPAVTL AHELIHAGHR LYGIAINPNR
VFKVNTNAYY EMSGLEVSFE ELRTFGGHDA KFIDSLQENE FRLYYYNKFK DIASTLNKAK
SIVGTTASLQ YMKNVFKEKY LLSEDTSGKF SVDKLKFDKL YKMLTEIYTE DNFVKFFKVL
NRKTYLNFDK AVFKINIVPK VNYTIYDGFN LRNTNLAANF NGQNTEINNM NFTKLKNFTG
LFEFYKLLCV RGIITSKAGA GKSLVPRGSA GAGALNDLCI KVNNWDLFFS PSEDNFTNDL
NKGEEITSDT NIEAAEENIS LDLIQQYYLT FNFDNEPENI SIENLSSDII GQLELMPNIE
RFPNGKKYEL DKYTMFHYLR AQEFEHGKSR IALTNSVNEA LLNPSRVYTF FSSDYVKKVN
KATEAAMFLG WVEQLVYDFT DETSEVSTTD KIADITIIIP YIGPALNIGN MLYKDDFVGA
LIFSGAVILL EFIPEIAIPV LGTFALVSYI ANKVLTVQTI DNALSKRNEK WDEVYKYIVT
NWLAKVNTQI DLIRKKMKEA LENQAEATKA IINYQYNQYT EEEKNNINFN IDDLSSKLNE
SINKAMININ KFLNQCSVSY LMNSMIPYGV KRLEDFDASL KDALLKYIYD NRGTLIGQVD
RLKDKVNNTL STDIPFQLSK YVDNQRLLST FTEYIKNIIN TSILNLRYES NHLIDLSRYA
SKINIGSKVN FDPIDKNQIQ LFNLESSKIE VILKNAIVYN SMYENFSTSF WIRIPKYFNS
ISLNNEYTII NCMENNSGWK VSLNYGEIIW TLQDTQEIKQ RVVFKYSQMI NISDYINRWI
FVTITNNRLN NSKIYINGRL IDQKPISNLG NIHASNNIMF KLDGCRDTHR YIWIKYFNLF
DKELNEKEIK DLYDNQSNSG ILKDFWGDYL QYDKPYYMLN LYDPNKYVDV NNVGIRGYMY
LKGPRGSVMT TNIYLNSSLY RGTKFIIKKY ASGNKDNIVR NNDRVYINVV VKNKEYRLAT
NASQAGVEKI LSALEIPDVG NLSQVVVMKS KNDQGITNKC KMNLQDNNGN DIGFIGFHQF
NNIAKLVASN WYNRQIEASS RTLGCSWEFI PVDDGWGERP LASPAAPAPA SPAAPAPSAP
AASPAAPAPA SPAAPAPSAP AASPAAPAPA SPAAPAPSAP AASPAAPAPA SPAAPAPSAP
AASPAAPAPA SPAAPAPSAP ALVPRSSHHH HHH
SEQ ID NO 3: recombinant single-chain precursor PAS100-BoNT/
A-R1273A-PAS100 including His.tag (nucleic acid sequence)
ATGGGTAGCAGCCATCATCATCATCACCATGGTAGCCTGGTTCCGCGTAGCTCTTCTGCA
AGTCCGGCAGCACCGGCACCGGCATCACCAGCAGCACCAGCACCTAGCGCACCGGCAGCA
AGCCCTGCTGCACCGGCACCAGCAAGTCCTGCAGCCCCTGCACCTTCAGCACCGGCAGCT
AGTCCAGCAGCGCCTGCTCCGGCATCTCCTGCTGCTCCAGCACCGAGTGCTCCGGCAGCT
TCTCCTGCAGCACCAGCCCCTGCATCTCCGGCAGCGCCTGCACCAAGTGCCCCTGCAGCA
AGTCCAGCCGCACCAGCGCCTGCAAGTCCTGCTGCGCCAGCTCCATCTGCGCCTGCAGCA
CCGTTTGTTAATAAACAGTTCAACTATAAAGATCCGGTGAACGGTGTTGATATCGCCTAT
ATCAAAATTCCGAATGCAGGTCAGATGCAGCCGGTTAAAGCCTTTAAAATCCATAACAAA
ATTTGGGTGATTCCGGAACGTGATACCTTTACCAATCCGGAAGAAGGTGATTTAAATCCG
CCTCCGGAAGCAAAACAGGTTCCGGTTAGCTATTATGATAGCACCTATCTGAGCACCGAT
AACGAGAAAGATAACTATCTGAAAGGTGTGACCAAACTGTTTGAACGCATTTATAGCACC
GATCTGGGTCGTATGCTGCTGACCAGCATTGTTCGTGGTATTCCGTTTTGGGGTGGTAGC
ACCATTGATACCGAACTGAAAGTTATTGATACCAATTGCATCAACGTGATTCAGCCGGAT
GGTAGCTATCGTAGCGAAGAACTGAATCTGGTTATTATTGGTCCGAGCGCAGATATCATT
CAGTTTGAATGTAAAAGCTTTGGCCACGAAGTTCTGAATCTGACCCGTAATGGTTATGGT
AGCACCCAGTATATTCGTTTTAGTCCGGATTTTACCTTTGGCTTTGAAGAAAGCCTGGAA
GTTGATACCAATCCGCTGCTGGGTGCAGGTAAATTTGCAACCGATCCGGCAGTTACCCTG
GCACATGAACTGATTCATGCAGGTCATCGTCTGTATGGTATTGCCATTAATCCGAATCGT
GTGTTCAAAGTGAATACCAACGCCTATTATGAAATGAGCGGTCTGGAAGTTAGCTTTGAA
GAACTGCGTACCTTTGGTGGTCATGATGCCAAATTTATCGATAGCCTGCAAGAAAATGAA
TTTCGCCTGTATTATTACAATAAATTCAAAGATATCGCCAGCACCCTGAACAAAGCAAAA
AGCATTGTTGGCACCACCGCAAGCCTGCAGTATATGAAAAATGTGTTTAAAGAAAAATAT
CTGCTGAGCGAAGATACCAGCGGTAAATTTAGCGTTGACAAACTGAAATTTGATAAACTG
TATAAAATGCTGACCGAAATCTACACCGAAGATAACTTCGTGAAATTTTTCAAAGTGCTG
AATCGCAAAACCTATCTGAACTTTGATAAAGCCGTGTTTAAAATCAATATTGTGCCGAAA
GTGAATTATACCATCTACGACGGCTTCAACTTAAGAAATACCAATCTGGCAGCCAATTTT
AACGGTCAGAATACCGAAATCAATAACATGAATTTTACCAAACTGAAAAACTTTACCGGT
CTGTTTGAATTCTACAAACTGCTGTGTGTGCGTGGTATTATTACCAGCAAAGCCGGTGCG
GGTAAAAGCCTGGTTCCTCGTGGTAGTGCGGGTGCGGGTGCACTGAATGATCTGTGTATT
AAAGTGAATAACTGGGACCTGTTTTTTAGCCCGAGTGAAGATAACTTTACCAATGATCTG
AACAAAGGCGAAGAAATTACCAGCGATACCAATATTGAAGCAGCCGAAGAAAACATTAGC
CTGGATCTGATTCAGCAGTACTATCTGACCTTTAACTTTGATAACGAGCCGGAAAACATC
AGCATTGAAAATCTGAGCAGCGATATTATTGGTCAGCTGGAACTGATGCCGAATATTGAA
CGTTTTCCGAACGGCAAAAAATACGAGCTGGATAAATACACCATGTTCCATTATCTGCGT
GCCCAAGAATTTGAACATGGTAAAAGCCGTATTGCACTGACCAATAGCGTTAATGAAGCA
CTGCTGAATCCGAGCCGTGTTTATACCTTTTTTAGCAGCGATTATGTGAAAAAAGTGAAT
AAAGCAACCGAGGCAGCAATGTTTCTGGGTTGGGTTGAACAGCTGGTTTATGATTTCACC
GATGAAACCAGCGAAGTTAGCACCACCGATAAAATTGCAGATATCACCATTATCATCCCG
TATATTGGTCCGGCACTGAATATTGGTAACATGCTGTATAAAGATGATTTCGTGGGTGCC
CTGATTTTTAGCGGTGCAGTTATTCTGCTGGAATTTATTCCGGAAATTGCCATTCCGGTT
CTGGGCACCTTTGCACTGGTTAGCTATATTGCAAATAAAGTTCTGACCGTGCAGACCATT
GATAATGCACTGAGCAAACGTAACGAAAAATGGGATGAGGTGTACAAATATATCGTGACC
AATTGGCTGGCCAAAGTTAATACCCAGATTGATCTGATCCGCAAAAAAATGAAAGAAGCC
CTGGAAAATCAGGCAGAAGCAACCAAAGCCATTATCAACTATCAGTATAACCAGTACACC
GAAGAAGAAAAAAACAATATTAACTTTAACATTGATGATCTGAGCAGCAAACTGAACGAG
AGCATTAACAAAGCCATGATTAACATTAACAAATTTCTGAATCAGTGCAGCGTGAGCTAT
CTGATGAATAGCATGATTCCGTATGGTGTGAAACGCCTGGAAGATTTTGATGCAAGCCTG
AAAGATGCACTGCTGAAATATATCTATGATAATCGTGGCACCCTGATTGGTCAGGTTGAT
CGTCTGAAAGATAAAGTGAACAATACCCTGAGTACCGATATTCCGTTTCAGCTGAGCAAA
TATGTTGATAATCAGCGTCTGCTGAGCACCTTTACCGAATATATCAAAAACATCATTAAT
ACCAGCATTCTGAACCTGCGCTATGAAAGCAATCATCTGATTGATCTGAGCCGTTATGCC
AGCAAAATCAACATTGGTAGCAAAGTGAACTTCGACCCGATTGATAAAAATCAGATTCAG
CTGTTTAACCTGGAAAGCAGCAAAATTGAAGTGATCCTGAAAAACGCCATCGTGTATAAT
AGCATGTATGAGAATTTCTCGACCAGCTTTTGGATTCGCATTCCGAAATATTTCAACAGC
ATTAGCCTGAACAACGAATATACCATCATTAATTGCATGGAAAACAATAGCGGTTGGAAA
GTGAGCCTGAATTATGGTGAAATTATCTGGACCCTGCAGGATACCCAAGAAATTAAACAG
CGTGTGGTGTTCAAATACAGCCAGATGATTAATATCAGCGACTATATCAACCGCTGGATC
TTTGTTACCATTACCAATAATCGCCTGAATAATAGCAAAATCTACATCAACGGTCGCCTG
ATTGATCAGAAACCGATTAGCAATCTGGGTAATATTCATGCCAGCAATAACATTATGTTC
AAACTGGATGGTTGCCGTGATACCCATCGTTATATTTGGATCAAATACTTTAACCTGTTT
GATAAAGAACTGAATGAAAAAGAAATTAAAGACCTGTATGATAATCAGAGCAACAGCGGC
ATTCTGAAAGATTTTTGGGGTGATTATCTGCAGTATGATAAACCGTATTACATGCTGAAT
CTGTATGATCCGAACAAATATGTGGATGTGAATAATGTGGGTATCCGTGGCTATATGTAT
CTGAAAGGACCGCGTGGTAGCGTTATGACCACCAACATTTATCTGAATAGCAGCCTGTAT
CGTGGCACCAAATTCATTATCAAAAAATACGCCAGCGGCAATAAAGATAACATTGTGCGT
AATAATGACCGCGTGTATATTAACGTTGTGGTGAAAAACAAAGAATATCGCCTGGCAACC
AATGCAAGCCAGGCAGGCGTTGAAAAAATTCTGAGCGCACTGGAAATTCCGGACGTCGGT
AATCTGAGCCAGGTTGTTGTTATGAAAAGCAAAAATGATCAGGGTATTACCAATAAATGC
AAAATGAACCTGCAGGACAATAACGGCAATGATATTGGTTTTATCGGCTTCCACCAGTTC
AACAATATTGCAAAACTGGTTGCCAGCAATTGGTATAATCGTCAGATTGAAGCTAGCAGC
CGTACCCTGGGTTGTAGCTGGGAATTTATCCCGGTTGATGATGGTTGGGGTGAACGTCCG
CTGGCCAGCCCTGCAGCGCCAGCACCGGCAAGCCCAGCTGCTCCTGCTCCTAGTGCACCA
GCAGCATCACCGGCTGCCCCTGCACCAGCTTCACCGGCAGCTCCAGCTCCGTCAGCTCCT
GCAGCTTCACCAGCTGCGCCTGCCCCTGCAAGCCCAGCAGCCCCAGCCCCATCAGCCCCT
GCTGCCTCACCAGCCGCTCCAGCCCCAGCATCCCCAGCCGCACCGGCACCGTCTGCTCCA
GCTGCTAGTCCTGCGGCTCCTGCCCCTGCCAGTCCTGCGGCACCAGCACCAAGTGCACCA
GCGCTGGTTCCGCGTAGCTCTCATCACCATCATCACCATTGAGATCTGGTGCCACGTGGT
AGCGCAAATTCGAGCTCCGTGGATAAACTGTGGTCCCATCCGCAGTTTGAAAAATGA

1. A recombinant single-chain precursor botulinum neurotoxin serotype A comprising at least one additional domain consisting of at least 50 amino acid residues selected from the group consisting of at least one proline, at least one alanine and at least one serine residue, wherein the single-chain precursor neurotoxin further comprises at least one cleavage site for a protease selected from the group consisting of thrombin, HRV3C, Tobacco Etch Virus protease, enterokinase and factor X and an amino acid modification located at position 1273 and/or 1274 within the heavy chain of the neurotoxin according to SEQ ID NO: 1.

2. The recombinant single-chain precursor botulinum neurotoxin of claim 1, wherein said at least one domain comprises an amino acid sequence consisting of between 50 and 500 amino acid residues, more particularly between 70 and 300 amino acid residues, particularly 100 amino acid residues, 150 amino acid residues, or 200 amino acid residues.

3. The recombinant single-chain precursor botulinum neurotoxin of claim 1, wherein said at least one domain is inserted at a position selected from (i) the N-terminus of the light chain of said recombinant neurotoxin; (ii) the C-terminus of the light chain of said recombinant neurotoxin; (iii) the N-terminus of the heavy chain of said recombinant neurotoxin; or (iv) the C-terminus of the heavy chain of said recombinant neurotoxin.

4. The recombinant single-chain precursor botulinum neurotoxin of claim 1, wherein the amino acid arginine at position 1273 according to SEQ ID NO: 1 is substituted by an amino acid selected from the group of alanine, methionine, glutamine, leucine, isoleucine, phenylalanine, threonine, valine, tyrosine and serine.

5. The recombinant single-chain precursor botulinum neurotoxin of claim 1, wherein the amino acid serine at position 1274 according to SEQ ID NO: 1 is substituted by amino acid selected from the group of aspartic acid, tyrosine, asparagine and glutamic acid.

6. The recombinant single-chain precursor botulinum neurotoxin of claim 1, wherein the neurotoxin comprises two domains consisting of at least one proline, at least one alanine and at least one serine residue.

7. The recombinant single-chain precursor botulinum neurotoxin of claim 1, wherein one domain is inserted at the N-terminus of the light chain of said recombinant neurotoxin and one domain is inserted at the C-terminus of the heavy chain of said recombinant neurotoxin.

8. A recombinant botulinum neurotoxin serotype A obtainable by cleaving the recombinant single-chain precursor neurotoxin according to claim 1 with a protease selected from the group consisting of thrombin, HRV3C, Tobacco Etch Virus protease, enterokinase and factor Xa.

9. The recombinant neurotoxin obtainable according to claim 8, wherein said recombinant neurotoxin shows an increased duration of effect relative to an identical neurotoxin without said domain(s).

10. The recombinant neurotoxin obtainable according to claim 8 for the use in the treatment of a disease requiring improved chemodenervation, wherein the recombinant neurotoxin causes an increased duration of effect relative to a wildtype botulinum neurotoxin serotype A.

11. A composition comprising the recombinant neurotoxin according to claim 8.

12. A pharmaceutical composition comprising the recombinant neurotoxin of claim 8.

13. Use of the recombinant neurotoxin of claim 8 for cosmetic treatment.

14. A method for the generation of a recombinant neurotoxin according to claim 8, comprising the steps of:
obtaining a recombinant nucleic acid sequence encoding a recombinant single-chain precursor neurotoxin by the insertion of at least one nucleic acid sequence encoding said PAS-domain into a nucleic acid sequence encoding a parental neurotoxin and
by modifying the nucleic acid sequence encoding a botulinum neurotoxin at position 1273 and/or 1274 of the heavy chain of the neurotoxin according to SEQ ID NO: 1,
by inserting at least one cleavage site for a protease selected from the group consisting of thrombin, HRV3C, Tobacco Etch Virus protease, enterokinase and factor Xa in the loop region,
by heterologously expressing said recombinant nucleic acid sequence in a host cell, particularly in a bacterial host cell, more particularly in an E. coli host cell,
by cleaving the recombinant single-chain precursor neurotoxin with a protease selected from the group consisting of thrombin, HRV3C, Tobacco Etch Virus protease, enterokinase and factor Xa.

15. A nucleic acid sequence encoding the recombinant single-chain precursor botulinum neurotoxin of claim 1.


SEQUENCE LISTING (3 sequences)

SEQ ID NO: 1 - protein, 1296 aa, Artificial Sequence
  re BoNT A including His.tag
MQFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 2 - protein, 1533 aa, Artificial Sequence
  re SC-BoNT/A-R1273A-PAS100 including His.tag
MGSSHHHHHHGSLVPRSSSASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAAPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKAGAGKSLVPRGSAGAGALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIEASSRTLGCSWEFIPVDDGWGERPLASPAAPAPASPAAPAPSAPAASPAAPAPASPAAPAPSAPAASPAAPAPASPAAPAPSAPAASPAAPAPASPAAPAPSAPAASPAAPAPASPAAPAPSAPALVPRSSHHHHHH

SEQ ID NO: 3 - DNA, 4677 bp, Artificial Sequence
  re SC-PAS100-BoNT/A-R1273A-PAS100 incl His.tag (nucleic acid
atgggtagcagccatcatcatcatcaccatggtagcctggttccgcgtagctcttctgcaagtccggcagcaccggcaccggcatcaccagcagcaccagcacctagcgcaccggcagcaagccctgctgcaccggcaccagcaagtcctgcagcccctgcaccttcagcaccggcagctagtccagcagcgcctgctccggcatctcctgctgctccagcaccgagtgctccggcagcttctcctgcagcaccagcccctgcatctccggcagcgcctgcaccaagtgcccctgcagcaagtccagccgcaccagcgcctgcaagtcctgctgcgccagctccatctgcgcctgcagcaccgtttgttaataaacagttcaactataaagatccggtgaacggtgttgatatcgcctatatcaaaattccgaatgcaggtcagatgcagccggttaaagcctttaaaatccataacaaaatttgggtgattccggaacgtgatacctttaccaatccggaagaaggtgatttaaatccgcctccggaagcaaaacaggttccggttagctattatgatagcacctatctgagcaccgataacgagaaagataactatctgaaaggtgtgaccaaactgtttgaacgcatttatagcaccgatctgggtcgtatgctgctgaccagcattgttcgtggtattccgttttggggtggtagcaccattgataccgaactgaaagttattgataccaattgcatcaacgtgattcagccggatggtagctatcgtagcgaagaactgaatctggttattattggtccgagcgcagatatcattcagtttgaatgtaaaagctttggccacgaagttctgaatctgacccgtaatggttatggtagcacccagtatattcgttttagtccggattttacctttggctttgaagaaagcctggaagttgataccaatccgctgctgggtgcaggtaaatttgcaaccgatccggcagttaccctggcacatgaactgattcatgcaggtcatcgtctgtatggtattgccattaatccgaatcgtgtgttcaaagtgaataccaacgcctattatgaaatgagcggtctggaagttagctttgaagaactgcgtacctttggtggtcatgatgccaaatttatcgatagcctgcaagaaaatgaatttcgcctgtattattacaataaattcaaagatatcgccagcaccctgaacaaagcaaaaagcattgttggcaccaccgcaagcctgcagtatatgaaaaatgtgtttaaagaaaaatatctgctgagcgaagataccagcggtaaatttagcgttgacaaactgaaatttgataaactgtataaaatgctgaccgaaatctacaccgaagataacttcgtgaaatttttcaaagtgctgaatcgcaaaacctatctgaactttgataaagccgtgtttaaaatcaatattgtgccgaaagtgaattataccatctacgacggcttcaacttaagaaataccaatctggcagccaattttaacggtcagaataccgaaatcaataacatgaattttaccaaactgaaaaactttaccggtctgtttgaattctacaaactgctgtgtgtgcgtggtattattaccagcaaagccggtgcgggtaaaagcctggttcctcgtggtagtgcgggtgcgggtgcactgaatgatctgtgtattaaagtgaataactgggacctgttttttagcccgagtgaagataactttaccaatgatctgaacaaaggcgaagaaattaccagcgataccaatattgaagcagccgaagaaaacattagcctggatctgattcagcagtactatctgacctttaactttgataacgagccggaaaacatcagcattgaaaatctgagcagcgatattattggtcagctggaactgatgccgaatattgaacgttttccgaacggcaaaaaatacgagctggataaatacaccatgttccattatctgcgtgcccaagaatttgaacatggtaaaagccgtattgcactgaccaatagcgttaatgaagcactgctgaatccgagccgtgtttataccttttttagcagcgattatgtgaaaaaagtgaataaagcaaccgaggcagcaatgtttctgggttgggttgaacagctggtttatgatttcaccgatgaaaccagcgaagttagcaccaccgataaaattgcagatatcaccattatcatcccgtatattggtccggcactgaatattggtaacatgctgtataaagatgatttcgtgggtgccctgatttttagcggtgcagttattctgctggaatttattccggaaattgccattccggttctgggcacctttgcactggttagctatattgcaaataaagttctgaccgtgcagaccattgataatgcactgagcaaacgtaacgaaaaatgggatgaggtgtacaaatatatcgtgaccaattggctggccaaagttaatacccagattgatctgatccgcaaaaaaatgaaagaagccctggaaaatcaggcagaagcaaccaaagccattatcaactatcagtataaccagtacaccgaagaagaaaaaaacaatattaactttaacattgatgatctgagcagcaaactgaacgagagcattaacaaagccatgattaacattaacaaatttctgaatcagtgcagcgtgagctatctgatgaatagcatgattccgtatggtgtgaaacgcctggaagattttgatgcaagcctgaaagatgcactgctgaaatatatctatgataatcgtggcaccctgattggtcaggttgatcgtctgaaagataaagtgaacaataccctgagtaccgatattccgtttcagctgagcaaatatgttgataatcagcgtctgctgagcacctttaccgaatatatcaaaaacatcattaataccagcattctgaacctgcgctatgaaagcaatcatctgattgatctgagccgttatgccagcaaaatcaacattggtagcaaagtgaacttcgacccgattgataaaaatcagattcagctgtttaacctggaaagcagcaaaattgaagtgatcctgaaaaacgccatcgtgtataatagcatgtatgagaatttctcgaccagcttttggattcgcattccgaaatatttcaacagcattagcctgaacaacgaatataccatcattaattgcatggaaaacaatagcggttggaaagtgagcctgaattatggtgaaattatctggaccctgcaggatacccaagaaattaaacagcgtgtggtgttcaaatacagccagatgattaatatcagcgactatatcaaccgctggatctttgttaccattaccaataatcgcctgaataatagcaaaatctacatcaacggtcgcctgattgatcagaaaccgattagcaatctgggtaatattcatgccagcaataacattatgttcaaactggatggttgccgtgatacccatcgttatatttggatcaaatactttaacctgtttgataaagaactgaatgaaaaagaaattaaagacctgtatgataatcagagcaacagcggcattctgaaagatttttggggtgattatctgcagtatgataaaccgtattacatgctgaatctgtatgatccgaacaaatatgtggatgtgaataatgtgggtatccgtggctatatgtatctgaaaggaccgcgtggtagcgttatgaccaccaacatttatctgaatagcagcctgtatcgtggcaccaaattcattatcaaaaaatacgccagcggcaataaagataacattgtgcgtaataatgaccgcgtgtatattaacgttgtggtgaaaaacaaagaatatcgcctggcaaccaatgcaagccaggcaggcgttgaaaaaattctgagcgcactggaaattccggacgtcggtaatctgagccaggttgttgttatgaaaagcaaaaatgatcagggtattaccaataaatgcaaaatgaacctgcaggacaataacggcaatgatattggttttatcggcttccaccagttcaacaatattgcaaaactggttgccagcaattggtataatcgtcagattgaagctagcagccgtaccctgggttgtagctgggaatttatcccggttgatgatggttggggtgaacgtccgctggccagccctgcagcgccagcaccggcaagcccagctgctcctgctcctagtgcaccagcagcatcaccggctgcccctgcaccagcttcaccggcagctccagctccgtcagctcctgcagcttcaccagctgcgcctgcccctgcaagcccagcagccccagccccatcagcccctgctgcctcaccagccgctccagccccagcatccccagccgcaccggcaccgtctgctccagctgctagtcctgcggctcctgcccctgccagtcctgcggcaccagcaccaagtgcaccagcgctggttccgcgtagctctcatcaccatcatcaccattgagatctggtgccacgtggtagcgcaaattcgagctccgtggataaactgtggtcccatccgcagtttgaaaaatga